(19) United States
(12) Patent Application Publication
(10) Pub. No.: US 2021/0060166 A1
(43) Pub. Date: Mar. 4, 2021

(54) Method for the preparation of a 1,7-bis (4-hydroxy-3-methoxyphenyl)-1,6-heptadiene-3,5-dione system in polysorbates for the formation of micelles in an aqueous medium aimed at the properties of the bioavailability of curcuminoids

(71) Applicants: Horacio Astudillo De La Vega, Mexico City (MX); Andrés Baca Ibañez, Mexico City (MX); Citlaly Gutierrez Rodelo, Mexico City (MX)

(72) Inventors: Horacio Astudillo De La Vega, Mexico City (MX); Andrés Baca Ibañez, Mexico City (MX); Citlaly Gutierrez Rodelo, Mexico City (MX)

(21) Appl. No.: 17/006,262

(22) Filed: Aug. 28, 2020

Related U.S. Application Data
(60) Provisional application No. 62/892,784, filed on Aug. 28, 2019.

(30) Foreign Application Priority Data
Jan. 31, 2020 (MX) MX/A/2019/008950

(51) Int. Cl. A61K 47/08; A61K 9/107; A61K 47/26

(52) U.S. Cl. CPC A61K 47/08; A61K 47/26; A61K 9/1075

(57) ABSTRACT

The invention relates to the preparation of a mixture of 1,7-bis (4-hydroxy-3-methoxyphenyl)-1,6-heptadien-3,5-dione in polysorbates that allows the formation of micelles in water loaded with 1,7-bis (4-hydroxy-3-methoxyphenyl)-1,6-heptadien-3,5-dione. With the developed formulation it is proposed to avoid bioavailability problems of this molecule, allowing to better demonstrate its benefits directed to systemic therapeutic effects, for example, anti-inflammatory drugs, and to reduce the use of drugs that cause direct or indirect irritation to the gastrointestinal mucosa, kidney damage, Cushing syndrome and/or immunosuppression.

### CROSS-REFERENCE TO RELATED APPLICATIONS

This application claims the benefit of U.S. Provisional Patent Application No. 62/892,784, filed Aug. 28, 2019 and Mexican Patent Application No. MX/a/2019/008950, filed Jan. 31, 2020, each of which is incorporated by reference in its entirety for all purposes.

### FIELD OF INVENTION

The present invention relates to the development of a 1,7-bis (4-hydroxy-3-methoxyphenyl)-1,6-heptadien-3,5-dione (curcumin) system in a non-ionic surfactant for micelle formation, specifically for pharmaceutical preparations containing curcuminoids, applied for the improvement of their ability to dissolve in aqueous media, avoiding solubility problems and thus increasing their bioavailability.

Curcumin is a molecule with major bioavailability problems, due to its low intrinsic activity, poor absorption, high metabolization, metabolite inactivity and rapid elimination of the body (Heger M, van Golen R F, Broekgaarden M, et al. The molecular basis for the pharmacokinetics and pharmacodynamics of curcumin and its metabolites in relation to cancer Pharmacol Rev. 2013; 66 (1): 222-307).

Several investigations have observed that when administering curcumin-containing extracts orally and measuring their presence in serum of animals and voluntary patients, the fraction of the extract that reaches systemic circulation to exert its effect, is at levels below 50% of the administered dose, showing a very low bioavailability. It has been observed that when administering the dose of 2 g/kg, only 0.006±0.005 μg/mL serum concentration can be found (Ammon H P, and Wahl M A. Pharmacology of Curcuma longa. Planta Med. 1991; 57 (1): 1-7), likewise, when administered from 4-8 g, concentrations of 0.4-3.6 μM were detected in the sera tested (Shen L, and Ji H F. The pharmacology of curcumin: is it the degradation products? Trends Mol Med. 2012; 18 (3): 138-144). It has also been observed that the administration of 10 or 12 g of curcumin shows blood levels of only 50.5 mg/mL (Mullaicharam A R, and Maheswaran A. “Pharmacological effects of curcumin.” International Journal of Nutrition, Pharmacology, Neurological Diseases 2012; 2 (2): 92-99) and 51.2 mg/mL respectively (Hewlings S J, and Kalman D S. Foods. Curcumin: A Review of Its' Effects on Human Health. 2017; 6 (10): E92).

### OBJECT OF THE INVENTION

1,7-bis (4-hydroxy-3-methoxyphenyl)-1,6-heptadien-3,5-dione (curcumin) belongs to a group of chemical compounds called curcuminoids, characterized by being phenolic compounds of low molecular weight and for being extracted from the rhizomes of the Curcuma longa plant. Curcumin interacts with several molecular targets, exerting antioxidant, anti-inflammatory, hypoglycemic, antitumor, antiviral, antimicrobial effects, among others (Gupta S C, Patchva S, Aggarwal B B. Therapeutic roles of curcumin: lessons learned from clinical trials. AAPS J. 2013; 15 (1): 195-218; Safavy A, Raisch K P, Mantena S, et al. Design and development of water-soluble curcumin conjugates as potential anticancer agents. J Med Chem. 2007; 50 (24): 6284-6288; Sun M, Su X, Ding B, et al. Advances in nanotechnology-based delivery systems for curcumin. Nanomedicine (Lond). 2012; 7 (7): 1085-1100; Vareed S K, Kakarala M, Ruffin M T, et al Pharmacokinetics of curcumin conjugate metabolites in healthy human subjects Cancer Epidemiol Biomarkers Prev. 2008; 17 (6): 1411-1417; Prasad S, Tyagi A K, Aggarwal B B. Recent developments in delivery, bioavailability, absorption, and metabolism of curcumin: the golden pigment from golden spice. Cancer Res Treat. 2014; 46 (1): 2-18; Batrakova E V, Bronich T K, Vetro J A, et al. Polymer micelles as drug carriers. Nanoparticulates as drug carriers. 2006: 57-93).

Multiple studies in animal models and humans have demonstrated the efficacy and safety of curcumin as a potential therapeutic agent (Kocher A, Schiborr C, Behnam D. The oral bioavailability of curcuminoids in healthy humans is markedly enhanced by micellar solubilization but not further improved by simultaneous ingestion of sesamin, ferulic acid, naringenin and xanthohumol. Journal of Functional Foods. 2015; 14: 183-191; Gong C, Deng S, Wu Q, et al. Improving antiangiogenesis and anti-tumor activity of curcumin by biodegradable polymeric micelles. Biomaterials 2013; 34 (4): 1413-1432). However, the molecule's physicochemical and pharmacokinetic properties limit its bioavailability when administered orally. It has been observed that between 40 and 70% of curcumin is not absorbed and results excreted in feces. On the other hand, once absorbed, its hepatic and intestinal metabolism is high and proceeds to be eliminated through urine and feces in the form of glucoronide conjugates (Zhang Q, Polyakov N E, Chistyachenko Y S, et al. Preparation of curcumin self-micelle solid dispersion with enhanced bioavailability and cytotoxic activity by mechanochemistry. Drug Deliv. 2018; 25 (1): 198-209; Cheng A L, Hsu C H, Lin J K, et al. Phase I clinical trial of curcumin, a chemopreventive agent, in patients with high-risk or pre-malignant lesions. Anticancer Res. 2001; 21 (4B): 2895-2900). The small amount of curcumin that is absorbed through first-pass metabolism, enters the bloodstream and is rapidly metabolized by the tissue (Garcea G, Berry D P, Jones D J, et al. Consumption of the putative chemopreventive agent curcumin by cancer patients: assessment of curcumin levels in the colorectum and their pharmacodynamic consequences Cancer Epidemiol Biomarkers Prev. 2005; 14 (1): 120-125).

In studies on colorectal tissue of healthy volunteers and cancer patients, it has been examined that after administering 3600 mg of curcumin, when evaluating serum levels, only 12.7±5.7 nM/g and 7.7±1.8 nM/g respectively are found. (Garcea G, Berry D P, Jones D J, et al. Consumption of the putative chemopreventive agent curcumin by cancer patients: assessment of curcumin levels in the colorectum and their pharmacodynamic consequences. Cancer Epidemiol Biomarkers Prev. 2005; 14 (1): 120-125). In another study conducted by the same research group, it was found that, in patients with colorectal cancer with liver metastasis, that where administrated 450 to 3600 mg of curcumin during the week prior to their operation, the assessment of serum levels found no circulating curcumin (Rahimi H R, Nedaeinia R, Sepehri Shamloo A, et al. Novel delivery system for natural products: Nano-curcumin formulations. Avicenna J Phytomed. 2016; 6 (4): 383-398).

During the last decade it has been shown that formulations based on nanometric structures such as micelles, increase water solubility and blood circulation time of hydrophobic drugs. Micellar structures are colloidal aggregates, thermodynamically stable, formed spontaneously by placing amphiphilic species, such as polysorbates in aqueous solutions. Polysorbates are sorbitan-derived liquids and are used to solubilize substances in water-based products. Commercial polysorbates are obtained from sorbitan by ethoxylation and esterification with fatty acids. (Anand P, Kunnumakkara A B, Newman R A, et al. Bioavailability of curcumin: problems and promises. Mol Pharm. 2007 November-December; 4 (6): 807-818).

William Griffin presented the Hydrophilic-Lipophilic Balance system (HLB) in the 1940's as a method of stablish which emulsifier would work best with the oil phase of an emulsified product, at the present time it is described as the balance of the size and strength of the hydrophilic and lipophilic moieties of a surfactant molecule. The HLB scale ranges from 0 to 20 (Griffin W C. Classification of Surface-Active Agents by “HLB”. J. Soc. Cosmet. Chem. 1949; 1: 311-326; Griffin W C. Calculation of HLB values of non-ionic surfactants, J. Soc. Cosmet. Chem. 1954; 5: 249-256).

The results of several studies of curcumin encapsulation in this type of structures coincide in that the solubility and pharmacokinetic behavior of the molecule, improve in several orders of magnitude compared to free curcumin, also enhancing its therapeutic effects in animal models and humans (Kakarala M, Brenner D E, Korkaya H. et al. Targeting breast stem cells with the cancer preventive compounds curcumin and piperine. Breast Cancer Res Treat. 2010; 122 (3): 777-785; 21. Suresh D, and Srinivasan K. Studies on the in vitro absorption of spice principles curcumin, capsaicin and piperine in rat intestines. Food Chem Toxicol. 2007 August; 45 (8): 1437-1442). The results of the studies on the absorption of free curcumin and micellar curcumin showed the advantages of the latter option with respect to free curcumin. In an in vitro model the absorption of curcumin in two formulations through inverted rat intestine was analyzed, the results showed a 9% increase in the absorption of encapsulated curcumin (Ma Z, Shayeganpour A, Brocks D R, et al High-performance liquid chromatography analysis of curcumin in rat plasma: application to pharmacokinetics of polymeric micellar formulation of curcumin Biomed Chromatogr. 2007; 21 (5): 546-552). It has also been found that polymeric micelles can increase the half-life of curcumin 60 times in the bloodstream of rats, when compared against solvated curcumin with PEG, N, N-Dimethylacetamide and dextrose. On the other hand, it has been shown that, when using a curcumin and phospholipid complex, 3 times better solubility and hepatoprotective effects are obtained contrasted to free curcumin in rats (Maiti K, Mukherjee K, Gantait A, et al. Curcumin-phospholipid complex: Preparation, therapeutic evaluation and pharmacokinetic study in rats. Int J Pharm. 2007; 330 (1-2): 155-163).

The state of the art found on the document ES2709653T3, shows a method for manufacturing a composition consisting essentially of curcumin mixed with a suitable portion of turmeric, to enhance the bioavailability of curcumin.

The document ES2511145T3 provides a curcuminoid formulation, comprising a nutritionally acceptable thermoplastic polymer, and a phosphatide that allow optimal bioavailability of curcumin.

The document MX2011010093A refers to a composition of fats, proteins and carbohydrates that include a combination of curcumin, demethoxycurcumin and bisdemethoxycurcumin, whose combination is solubilized in a polar oil with values of 0.7 and 0.14 hydrophilic-lipophilic balance for the increase of bioavailability.

The document US20160151440 comprises a composition with a natural emulsifier isolated from the American tree Quillaja saponaria and lecithin, without synthetic polymers; to increase the bioavailability of curcumin because dietary fibers retain curcumin for a prolonged time.

The document US20190105287 refers to curcumin methods and compositions encapsulated in a colloidal and/or liposomal drug delivery system for the treatment or prevention of cancer.

The present invention provides a method consisting of the mixture of 1,7-bis (4-hydroxy-3-methoxyphenyl)-1,6-heptadien-3,5-dione) with polysorbates, when added to an aqueous system, micelles are formed encapsulating the curcumin, this facilitates administration, dose accuracy and bioavailability of curcuminoids.

### BRIEF DESCRIPTION OF THE DRAWINGS

FIG. 1. Relationship of concentration of 1,7-bis (4-hydroxy-3-methoxyphenyl)-1,6-heptadien-3,5-dione (mg/L) in solution compared to the percentage of polysorbates (% w/w).

FIG. 2. Evaluation of the presence or absence of analyte precipitate (1,7-bis (4-hydroxy-3-methophenyl)-1,6-heptadien-3,5-dione) in mixtures with different concentrations of polysorbates (mg/mL).

FIG. 3A-FIG. 3D. The HLB value of polysorbates in the invention.

FIG. 4. Concentration of 1,7-bis (4-hydroxy-3-methoxyphenyl)-1,6-heptadien-3,5-dione (mg/L) over time (h) in the presence of polysorbates.

FIG. 5. Concentration of 1,7-bis (4-hydroxy-3-methoxyphenyl)-1,6-heptadien-3,5-dione (mg/L) over time (h) in the medium in the absence of polysorbates.

FIG. 6. Concentration of 1,7-bis (4-hydroxy-3-methoxyphenyl)-1,6-heptadien-3,5-dione (mg/L) compared to the concentration of polysorbates (%).

FIG. 7. Comparison in the concentration of 1,7-bis (4-hydroxy-3-methoxyphenyl)-1,6-heptadien-3,5-dione (mg/L) released in the absence and presence of polysorbates for 20 hours.

### DETAILED DESCRIPTION OF THE INVENTION

To evaluate the encapsulation behavior of 1,7-bis (4-hydroxy-3-methoxyphenyl)-1,6-heptadien-3,5-dione in micelles formed from polysorbates in an aqueous medium, experiments were conducted to evaluate the micellar integration capacity of 1,7-bis (4-hydroxy-3-methoxyphenyl)-1,6-heptadien-3,5-dione.

The system consisted of a mixture of 1,7-bis (4-hydroxy-3-methoxyphenyl)-1,6-heptadien-3,5-dione) with polysorbates of different values of HLB, which, when added to an aqueous system, conduced to the formation of micelles with curcumin integrated inside. Next, the method of preparing the mixture of the invention is described:

1.—The 1,7-bis (4-hydroxy-3-methoxyphenyl)-1,6-heptadien-3,5-dione was added to the heaviest polysorbates (named after its greater capacity for suspend curcumin).
2.—The suspension was mixed until the absence of 1,7-bis (4-hydroxy-3-methoxyphenyl)-1,6-heptadien-3,5-dione clusters.
3.—The suspension was subjected to sonication, filtration and nitrogen pumping for degassing the mixture.
4.—The suspension was mixed with light polysorbates (with a smaller value of HLB classified as having a lower capacity to suspend curcumin) to control the viscosity of the mixture, facilitating the administration of the mixture of 1,7-bis (4-hydroxy-3-methoxyphenyl)-1,6-heptadien-3,5-dione and favor dose accuracy.

### Example 1. Determination of the Maximum Amount of Curcumin that is Integrated into an Aqueous Phase with the Use of Polysorbates

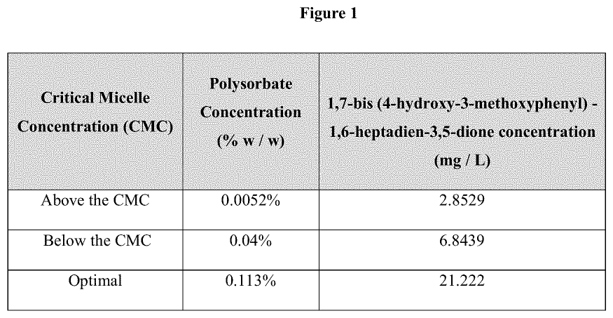

Integration into the micellar system of 1,7-bis (4-hydroxy-3-methoxyphenyl)-1,6-heptadien-3,5-dione present in suspensions was analyzed. Three concentrations of polysorbate of different value of HLB were evaluated in a 1:1 ratio for each mixture; With this combination of surfactants, 3 aqueous systems were prepared at concentrations of 0.0052%, 0.04% and 0.113% (% weight/weight). The first corresponds to a concentration below the individual Critical Micellar Concentration (CMC) of each surfactant 2.8529 mg/L, the second corresponds to a concentration slightly above the CMC 6.8439 mg/L and the third corresponds to a concentration well above the CMC, respectively 21.222 mg/L (FIG. 1). The method consisted of weighing 57.57 mg of 1,7-bis (4-hydroxy-3-methoxyphenyl)-1,6-heptadien-3,5-dione, to which 250 mL of the 3 concentrations mentioned were separately added. After 30 minutes of stirring at 500 RPM at 25° C., the suspensions were allowed to stand for 30 minutes and a sample from the supernatant was taken for UV/VIS spectrophotometric evaluation at 424 nm, using the 3 mentioned concentrations as blank.

Results:

Through the tests described, the ability of the polysorbates to incorporate 1,7-bis (4-hydroxy-3-methoxyphenyl)-1,6-heptadien-3,5-dione into the micellar system were examined.

Result 1

The ability to form a micellar system of 1,7-bis (4-hydroxy-3-methoxyphenyl)-1,6-heptadien-3,5-dione at different percentages of polysorbates was evaluated. The best curcumin integration result was obtained when the polysorbate concentration reached 0.113% as shown in FIG. 1, from the absorbances obtained, the amount of 1,7-bis (4-hydroxy-3-methoxyphenyl)-1,6-heptadien-3,5-dione in suspension was quantified, interpolating the data with a calibration curve designed from solutions of 1,7-bis (4-hydroxy-3-methoxyphenyl)-1,6-heptadien-3,5-dione in polysorbates, of known concentration and using the 3 mentioned concentrations of polysorbates without the 1,7-bis (4-hydroxy-3-methoxyphenyl)-1,6-heptadien-3,5-dione analyte as a blank.

The concentration of 1,7-bis (4-hydroxy-3-methoxyphenyl)-1,6-heptadien-3,5-dione in solution is higher as the percentage of polysorbates increases. At a concentration of polysorbates above the CMC, a curcumin concentration of 6.8439 mg/L is observed, in contrast to 2.8529 mg/L that dissolves when the experimental concentration is below the CMC (FIG. 1).

On the other hand, when the concentration of polysorbates is 0.113% which corresponds to the present invention, the integration capacity of 1,7-bis (4-hydroxy-3-methoxyphenyl)-1,6-heptadien-3,5-dione reaches a concentration of 21. 222 mg/L (FIG. 1).

Result 2

The suspension capacity of the polysorbates for 1,7-bis (4-hydroxy-3-methoxyphenyl)-1,6-heptadien-3,5-dione was evaluated by gravimetry according to PHARMACOPEA OF THE UNITED MEXICAN STATES (FEUM), volume 1, eleventh edition.

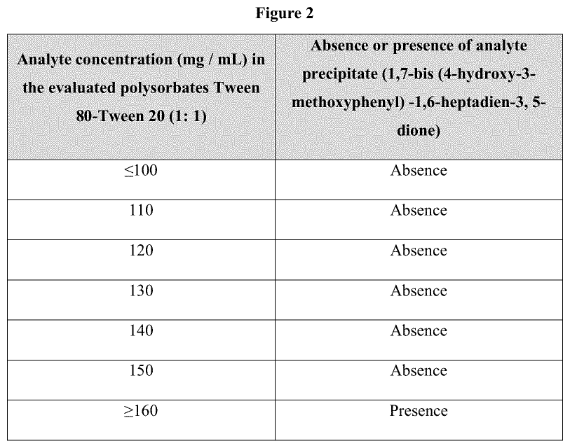

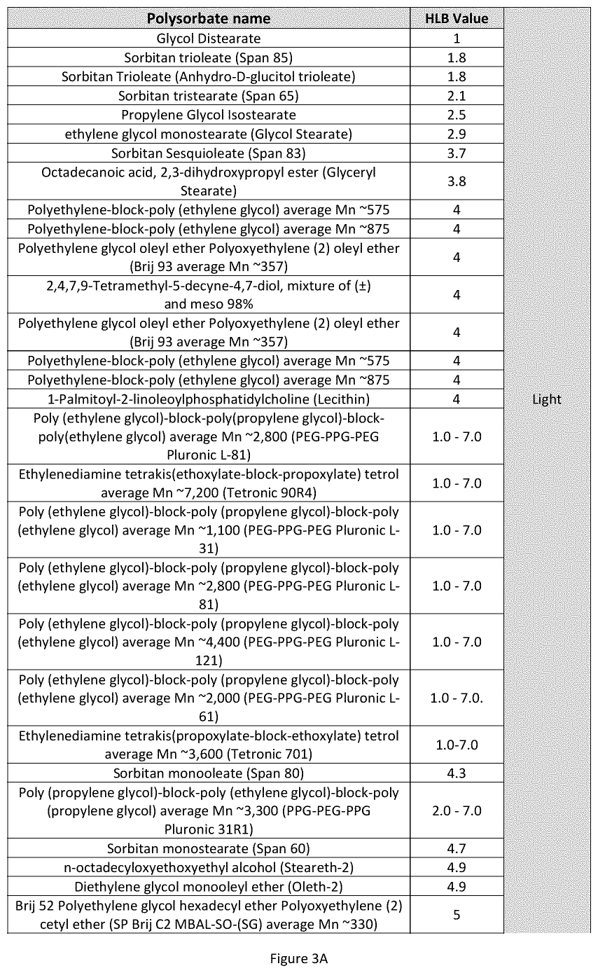

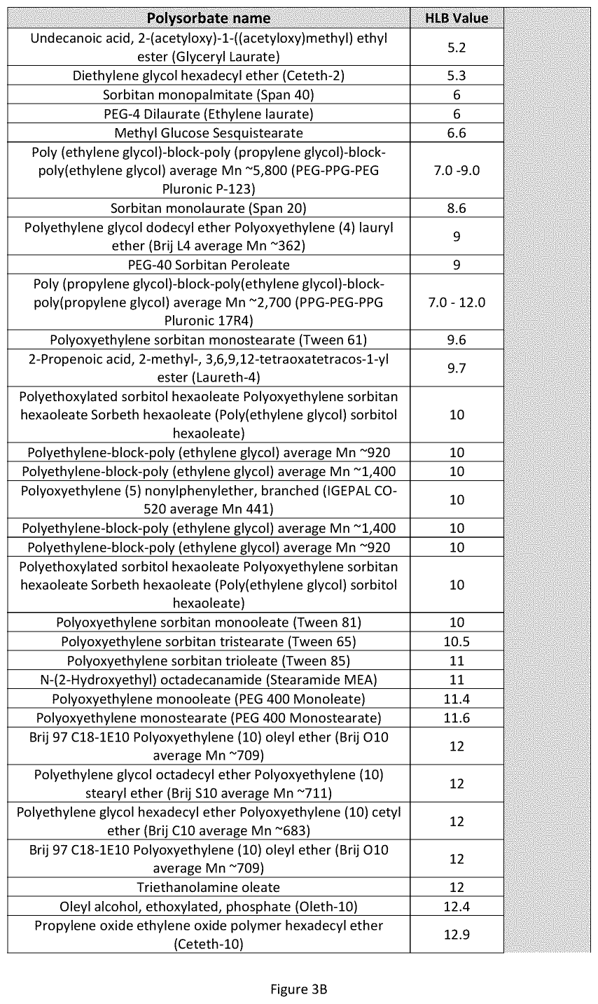

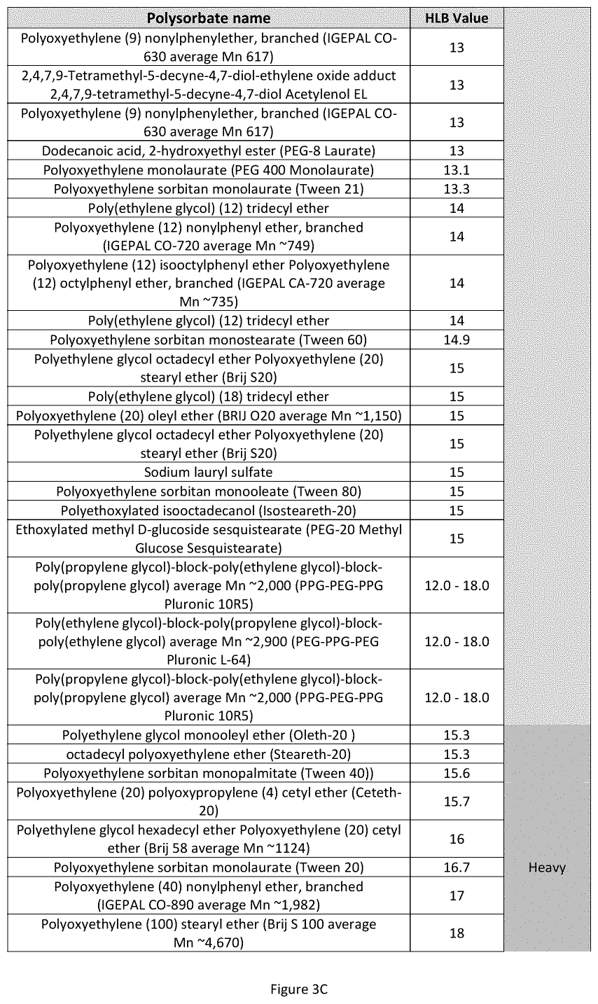

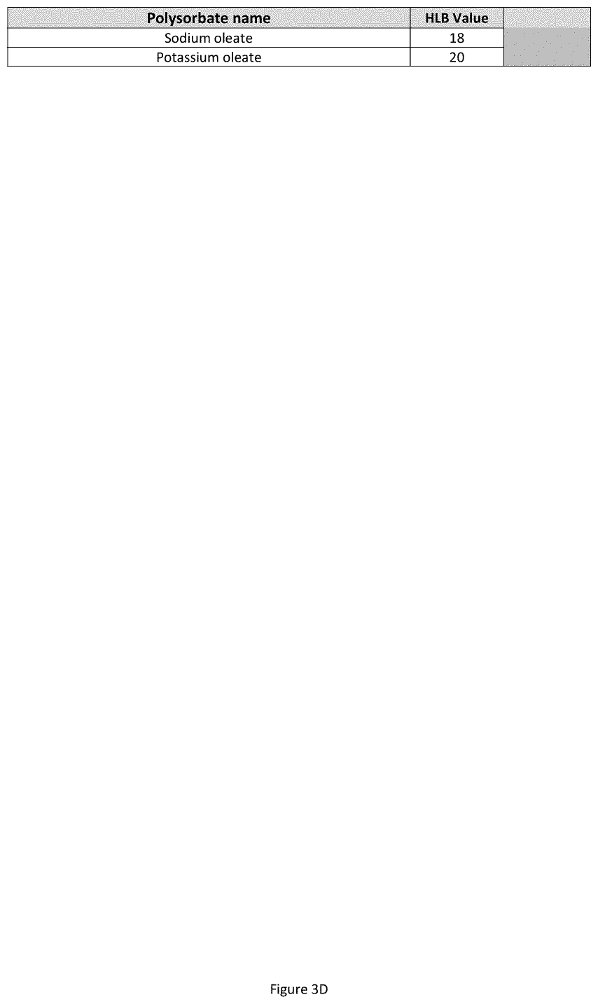

Known concentrations of 2-[2-[3,4-bis(2-hydroxyethoxy)oxolan-2-yl]-2-(2-hydroxyethoxy)ethoxy]ethyl dodecanoate (Polysorbate 20) or Tween 20 and 2-[2-[3,5-bis(2-hydroxyethoxy)oxolan-2-yl]-2-(2-hydroxyethoxy)ethoxy]ethyl(E)-octadec-9-enoate (Polysorbate 80) or Tween 80 were used to achieve a known final concentration of 1,7-bis (4-hydroxy-3-methoxyphenyl)-1,6-heptadien-3,5-dione (analyte). Stating that at ratios of <100 of Tween 20 or 80, the capacity is not sufficient to suspend the required analyte dose. However, after 160 mg/mL (1:1) the presence of precipitate was found. In FIG. 2, different concentrations of polysorbate (Tween 80-Tween 20 (1:1) are specified in which, when mixed with analyte results in absence or presence of analyte precipitate (FIG. 3).

FIG. 3 shows the HLB value of the polysorbates in the invention depending on their specific physicochemical properties, in which it is possible to suspend 1,7-bis (4-hydroxy-3-methoxyphenyl)-1.6-heptadien-3,5-diona. From the HLB values of the polysorbates, it can be determined which of them has a better ability to suspend curcumin. The higher the HLB the better it is at suspending curcumin, for this example, the Lauric acid (Tween20) (FIG. 3).

However, a combination of multiple polysorbates is necessary since the use of polysorbates with high HLB value tends to coincide with high viscosity. This makes the dose accuracy harder. Thus, a second polysorbate of lower viscosity is needed.

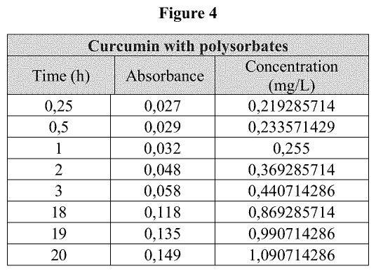

A dialysis experiment was performed to see the release of curcumin molecules from the solution (FIG. 4).

Spectrum™ Spectra/Por™ 4 RC Dialysis Membrane Tubing 12,000 to 14,000 Dalton MWCO, cellulose, was used. The release conditions were pH 6.8, Polysorbate 1, 8%, Polysorbate 2, 4%. The pH 6.8 was used as Kiyohiko Sugano, Hirokazu Hamada, Minoru Machida, Hidetoshi Ushio, Kimitoshi Saitoh, Katsuhide Terada. Optimized conditions of bio-mimetic artificial membrane permeation assay. International Journal of Pharmaceutics Volume 228, Issues 1-2, 9 Oct. 2001, Pages 181-188 to simulate the conditions of the intestine, where the absorption of the compound is carried out. Concentrations of 8 and 4 percent of the polysorbates were used to ensure that the solution is above the CMC and the ability to form micelles is not lost, since the analytical method requires that an amount of release medium gets removed and replenish with simple medium (FEUM, volume 1, eleventh edition.). As it is shown in FIG. 4, it is observed that the absorbance and thus concentration of 1,7-bis (4-hydroxy-3-methoxyphenyl)-1,6-heptadien-3,5-dione, with polysorbates increases with time from 0.25 h to 20 h.

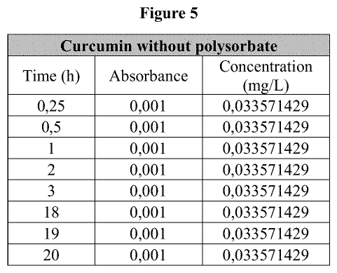

On the other hand, FIG. 5 shows that, the release of curcumin does not show any increase in concentration over time in the absence of polysorbates in the medium. This indicates that the presence of polysorbates is needed for the integration of 1,7-bis (4-hydroxy-3-methoxyphenyl)-1,6-heptadien-3,5-dione into the micellar system. For example, at 20 hours the concentration of 1,7-bis (4-hydroxy-3-methoxyphenyl)-1,6-heptadien-3,5-dione is 3 times higher in the experiment conducted with polysorbates than the one performed in the absence of these.

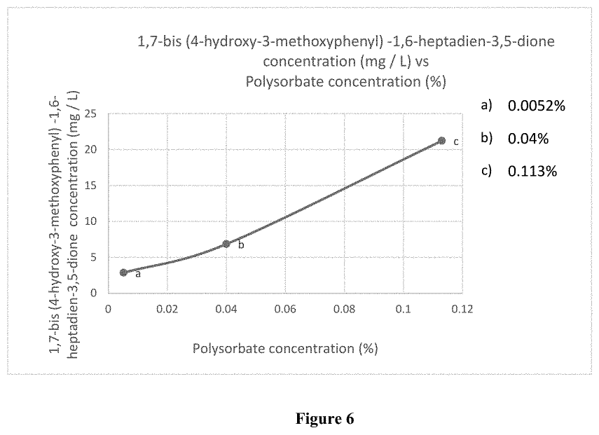

FIG. 6 shows the increase in concentration of 1,7-bis (4-hydroxy-3-methoxyphenyl)-1,6-heptadien-3,5-dione encapsulated as the percentage of polysorbates increases (0.0052%, 0.04% and 0.113%) in the solution. The percentage of polysorbates at which curcumin is below the CMC is shown in point a), in b) the percentage of polysorbates is shown slightly above the CMC and in c) above the CMC. The foregoing illustrates that the greater the number of micelles, the greater the capacity of incorporation of curcumin into the system.

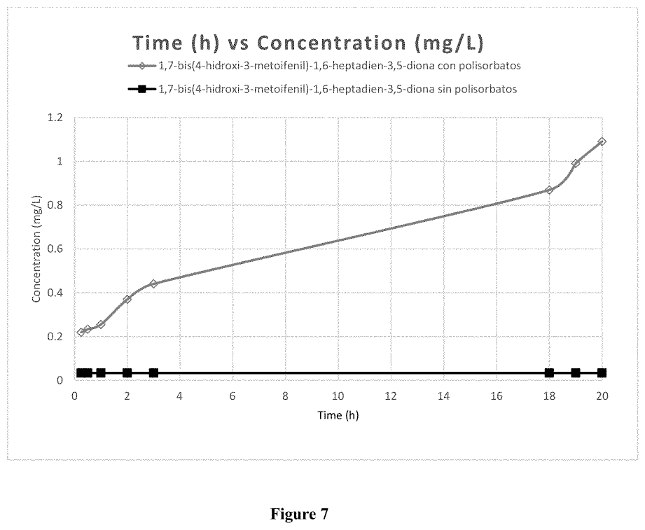

To evaluate the comparison of a system with polysorbates (gray) and a system without polysorbates (bold) over time, FIG. 7 represents the release through the cellulose membrane of curcumin, that can be observed when there are polysorbates in the system from 0 to 20 hours. The concentration of 1,7-bis (4-hydroxy-3-methoxyphenyl)-1,6-heptadien-3,5-dione in solution without polysorbates does not change with time thus showing the need of polysorbates to increase the analytes concentration.

1. A method for the development of a suspension of 1,7-bis (4-hydroxy-3-methoxyphenyl)-1,6-heptadien-3,5-dione in polysorbates for the formation of micelles in aqueous medium directed to the bioavailability properties of curcuminoids.

2. A manufacturing method in accordance with claim 1, containing at least one of the following curcuminoids: 1,7-bis (4-hydroxy-3-methoxyphenyl)-1,6-heptadiene-3,5-dione, Acid (2S,3 S,4S,5R,6S)-3,4,5-Trihydroxy-6-(4-((1E,4Z,6E)-5-hydroxy-7-(4-hydroxy-3-hydroxymethoxyphenyl)-3-oxohepta-1,4,6-trien-1-yl)-2-methoxyphenoxy)tetrahydro-2H-pyrano-2-oic, (E)-1,7-di(4-hydroxy-3-methoxyphenyl) hepta-1-eno-3,5-dione, 1,7-di(4-hydroxy-3-methoxyphenyl) heptane-3,5-dione, [4-[(1E,6E)-7-(4-hydroxy-3-methoxyphenyl)-3,5-dioxohepta-1,6-dienyl]-2-methoxyphenyl]hydrogen sulfate, 5-hydroxy-1,7-di(4-hydroxy-3-methoxyphenyl)hepta-3-one, 2-(4-hydroxy-3-methoxyphenyl)-6-[€-2-(4-hydroxy-3-methoxyphenyl)ethenyl]-2,3-dihydropyran-4-one or (1E,6E)-1,7-di (4-hydroxyphenyl)hepta-1,6-diene-3,5-dione.

3. A method in accordance with claim 1, containing at least some of the polysorbates that are considered heavy, meaning they possess a high capability to suspend curcuminoids: Polyethylene glycol monooleyl ether (Oleth-20), octadecyl polyoxyethylene ether (Steareth-20), Polyoxyethylene sorbitan monopalmitate (Tween 40)), Polyoxyethylene (20) polyoxypropylene (4) cetyl ether (Ceteth-20), Polyethylene glycol hexadecyl ether Polyoxyethylene (20) cetyl ether (Brij 58 average Mn ˜1124), Polyoxyethylene sorbitan monolaurate (Tween 20), Polyoxyethylene (40) nonylphenyl ether, branched (IGEPAL CO-890 average Mn ˜1,982), Polyoxyethylene (100) stearyl ether (Brij S 100 average Mn ˜4,670), Sodium oleate, Potassium oleate.

4. A method in accordance with claim 1, containing polysorbates that are considered light, meaning they possess little capability to suspend curcuminoids: Glycol Distearate, Sorbitan trioleate (Span 85), Sorbitan Trioleate (Anhydro-D-glucitol trioleate), Sorbitan tristearate (Span 65), Propylene Glycol Isostearate, ethylene glycol monostearate (Glycol Stearate), Sorbitan Sesquioleate (Span 83), Octadecanoic acid, 2,3-dihydroxypropyl ester (Glyceryl Stearate), Polyethylene-block-poly (ethylene glycol) average Mn ˜575, Polyethylene-block-poly (ethylene glycol) average Mn ˜875, Polyethylene glycol oleyl ether Polyoxyethylene (2) oleyl ether (Brij 93 average Mn ˜357), 2,4,7,9-Tetramethyl-5-decyne-4,7-diol, mixture of (±) and meso 98%, Polyethylene glycol oleyl ether Polyoxyethylene (2) oleyl ether (Brij 93 average Mn ˜357), Polyethylene-block-poly (ethylene glycol) average Mn ˜575, Polyethylene-block-poly (ethylene glycol) average Mn ˜875, 1-Palmitoyl-2-linoleoylphosphatidylcholine (Lecithin), Poly (ethylene glycol)-block-poly(propylene glycol)-block-poly(ethylene glycol) average Mn ˜2,800 (PEG-PPG-PEG Pluronic L-81), Ethylenediamine tetrakis(ethoxylate-block-propoxylate) tetrol average Mn ˜7,200 (Tetronic 90R4), Poly (ethylene glycol)-block-poly (propylene glycol)-block-poly (ethylene glycol) average Mn ˜1,100 (PEG-PPG-PEG Pluronic L-31), Poly (ethylene glycol)-block-poly (propylene glycol)-block-poly (ethylene glycol) average Mn ˜2,800 (PEG-PPG-PEG Pluronic L-81), Poly (ethylene glycol)-block-poly (propylene glycol)-block-poly (ethylene glycol) average Mn ˜4,400 (PEG-PPG-PEG Pluronic L-121), Poly (ethylene glycol)-block-poly (propylene glycol)-block-poly (ethylene glycol) average Mn ˜2,000 (PEG-PPG-PEG Pluronic L-61), Ethylenediamine tetrakis(propoxylate-block-ethoxylate) tetrol average Mn 3,600 (Tetronic 701), Sorbitan monooleate (Span 80), Poly (propylene glycol)-block-poly (ethylene glycol)-block-poly (propylene glycol) average Mn ˜3,300 (PPG-PEG-PPG Pluronic 31R1), Sorbitan monostearate (Span 60), n-ctadecyloxyethoxyethyl alcohol (Steareth-2), Diethylene glycol monooleyl ether (Oleth-2), Brij 52 Polyethylene glycol hexadecyl ether Polyoxyethylene (2) cetyl ether (SP Brij C2 MBAL-SO-(SG) average Mn ˜330), Undecanoic acid, 2-(acetyloxy)-1-((acetyloxy)methyl) ethyl ester (Glyceryl Laurate), Diethylene glycol hexadecyl ether (Ceteth-2), Sorbitan monopalmitate (Span 40), PEG-4 Dilaurate (Ethylene laurate), Methyl Glucose Sesquistearate, Poly (ethylene glycol)-block-poly (propylene glycol)-block-poly(ethylene glycol) average Mn ˜5,800 (PEG-PPG-PEG Pluronic P-123), Sorbitan monolaurate (Span 20), Polyethylene glycol dodecyl ether Polyoxyethylene (4) lauryl ether (Brij L4 average Mn ˜362), PEG-40 Sorbitan Peroleate, Poly (propylene glycol)-block-poly(ethylene glycol)-block-poly(propylene glycol) average Mn ˜2,700 (PPG-PEG-PPG Pluronic 17R4), Polyoxyethylene sorbitan monostearate (Tween 61), 2-Propenoic acid, 2-methyl-, 3,6,9,12-tetraoxatetracos-1-yl ester (Laureth-4), Polyethoxylated sorbitol hexaoleate Polyoxyethylene sorbitan hexaoleate Sorbeth hexaoleate (Poly(ethylene glycol) sorbitol hexaoleate), Polyethylene-block-poly (ethylene glycol) average Mn ˜920, Polyethylene-block-poly (ethylene glycol) average Mn ˜1,400, Polyoxyethylene (5) nonylphenylether, branched (IGEPAL CO-520 average Mn 441), Polyethylene-block-poly (ethylene glycol) average Mn ˜1,400, Polyethylene-block-poly (ethylene glycol) average Mn ˜920, Polyethoxylated sorbitol hexaoleate Polyoxyethylene sorbitan hexaoleate Sorbeth hexaoleate (Poly(ethylene glycol) sorbitol hexaoleate), Polyoxyethylene sorbitan monooleate (Tween 81), Polyoxyethylene sorbitan tristearate (Tween 65), Polyoxyethylene sorbitan trioleate (Tween 85), N-(2-Hydroxyethyl) octadecanamide (Stearamide MEA), Polyoxyethylene monooleate (PEG 400 Monoleate), Polyoxyethylene monostearate (PEG 400 Monostearate), Brij 97 C18-1E10 Polyoxyethylene (10) oleyl ether (Brij O10 average Mn ˜709), Polyethylene glycol octadecyl ether Polyoxyethylene (10) stearyl ether (Brij S10 average Mn ˜711), Polyethylene glycol hexadecyl ether Polyoxyethylene (10) cetyl ether (Brij C10 average Mn ˜683), Brij 97 C18-1E10 Polyoxyethylene (10) oleyl ether (Brij 010 average Mn ˜709), Triethanolamine oleate, Oleyl alcohol, ethoxylated, phosphate (Oleth-10), Propylene oxide ethylene oxide polymer hexadecyl ether (Ceteth-10), Polyoxyethylene (9) nonylphenylether, branched (IGEPAL CO-630 average Mn 617), 2,4,7,9-Tetramethyl-5-decyne-4,7-diol-ethylene oxide adduct 2,4,7,9-tetramethyl-5-decyne-4,7-diol Acetylenol EL, Polyoxyethylene (9) nonylphenylether, branched (IGEPAL CO-630 average Mn 617), Dodecanoic acid, 2-hydroxyethyl ester (PEG-8 Laurate), Polyoxyethylene monolaurate (PEG 400 Monolaurate), Polyoxyethylene sorbitan monolaurate (Tween 21), Poly(ethylene glycol) (12) tridecyl ether, Polyoxyethylene (12) nonylphenyl ether, branched (IGEPAL CO-720 average Mn ˜749), Polyoxyethylene (12) isooctylphenyl ether Polyoxyethylene (12) octylphenyl ether, branched (IGEPAL CA-720 average Mn ˜735), Poly(ethylene glycol) (12) tridecyl ether, Polyoxyethylene sorbitan monostearate (Tween 60), Polyethylene glycol octadecyl ether Polyoxyethylene (20) stearyl ether (Brij S20), Poly(ethylene glycol) (18) tridecyl ether, Polyoxyethylene (20) oleyl ether (BRIJ 020 average Mn ˜1,150), Polyethylene glycol octadecyl ether Polyoxyethylene (20) stearyl ether (Brij S20), Sodium lauryl sulfate, Polyoxyethylene sorbitan monooleate (Tween 80), Polyethoxylated isooctadecanol (Isosteareth-20), Ethoxylated methyl D-glucoside sesquistearate (PEG-20 Methyl Glucose Sesquistearate), Poly(propylene glycol)-block-poly(ethylene glycol)-block-poly(propylene glycol) average Mn ˜2,000 (PPG-PEG-PPG Pluronic 10R5), Poly(ethylene glycol)-block-poly(propylene glycol)-block-poly(ethylene glycol) average Mn ˜2,900 (PEG-PPG-PEG Pluronic L-64), Poly(propylene glycol)-block-poly(ethylene glycol)-block-poly(propylene glycol) average Mn ˜2,000 (PPG-PEG-PPG Pluronic 10R5).

5. A method in accordance with claim 1, characterized by firstly carrying out the addition of a polysorbate with a high capability to suspend curcuminoids to the total suspension, in accordance with claim 3.

6. A method in accordance with claim 1, characterized by incorporating curcuminoids into the suspension after a polysorbate with a high capability to suspend curcuminoids, a polysorbate with low or zero capability to suspend the curcuminoid in accordance with claim 4.

7. A method in accordance with claim 1, characterized by using a degassing procedure by filter medium, nitrogen or helium pumping, by vacuum, by membranes, stand, sonication and/or mixed at moderate temperatures (10° C. to 60° C.) after the suspension with polysorbates.

8. A method in accordance with the claim 2 that contains between 0.1 to 500 mg/mL of curcuminoids.

9. A method according to claim 3, wherein the polysorbates considered as heavy are integrated into the suspension in a concentration of 20-80% with respect to the polysorbates considered light.
